(19) United States
(12) Patent Application Publication
(10) Pub. No.: US 2013/0184584 A1
(43) Pub. Date: Jul. 18, 2013

(54) SYSTEMS AND METHODS FOR COMPUTERIZED ULTRASOUND IMAGE INTERPRETATION AND LABELING

(71) Applicant: Richard E. Berkey, Portland, OR (US)

(72) Inventor: Richard E. Berkey, Portland, OR (US)

(21) Appl. No.: 13/743,490

(22) Filed: Jan. 17, 2013

Related U.S. Application Data
(60) Provisional application No. 61/587,540, filed on Jan. 17, 2012.

(51) Int. Cl. A61B 8/08; A61B 8/12; A61B 8/06; A61B 8/10; A61B 8/00; A61B 8/02

(52) U.S. Cl. CPC A61B 8/5292; A61B 8/488; A61B 8/4444; A61B 8/54; A61B 8/463; A61B 8/02; A61B 8/06; A61B 8/10; A61B 8/12; A61B 8/565; A61B 8/467; A61B 8/486
USPC 600/441

(57) ABSTRACT

A system for labeling medical ultrasound images includes a processor, an ultrasound probe, a display screen, an ultrasound image database, and a labeling module. The display screen is configured to display ultrasound images collected by the ultrasound probe. The ultrasound image database includes a plurality of stored ultrasound images. The labeling module is configured to compare the ultrasound images displayed on the display screen to the stored ultrasound images and automatically label the ultrasound images displayed on the display screen.

### CROSS-REFERENCE TO RELATED APPLICATION

This application claims the benefit of the filing date of U.S. Provisional Application No. 61/587,540, filed Jan. 17, 2012, and entitled SYSTEMS AND METHODS FOR COMPUTERIZED ULTRASOUND IMAGE INTERPRETATION AND LABELING, the disclosure of which is incorporated, in its entirety, by reference.

### TECHNICAL FIELD

The present application related to medical ultrasound imaging, and more particularly relates to systems and methods for interpreting and labeling medical ultrasound images.

### BACKGROUND

The use of medical ultrasound has evolved far beyond its original application as a diagnostic radiology study. Advances have led to its use as a bedside exam that serves a critical role in patient care, particularly for critically ill or injured patients, for whom important clinical questions need to be answered quickly and accurately. The specialty of Emergency Medicine has led the integration of bedside ultrasound into clinical practice. In fact, ACEP (American College of Emergency Physicians) has published emergency ultrasound guidelines, establishing core and emerging applications for its use, emphasizing its critical role in modern medical care. ACGME (Accreditation Council for Graduate Medical Education) requires Emergency Medicine training program graduates to demonstrate competency in bedside ultrasound. It is expected that use of ultrasound will expand in primary care settings, in remote areas, and in developing countries.

Despite its numerous benefits (increased patient safety, improved quality and efficiency of care, reduction in complication rates of invasive procedures, reduced exposure to harmful ionizing radiation by decreasing need for computerized tomography, and cost savings), use of ultrasound does have limits and disadvantages. The method is operator-dependent, and requires skill and experience to acquire quality images and to interpret them with accuracy. As more providers with less training and experience (and no mandated demonstration of competency) begin to perform and interpret bedside ultrasound, new technology to overcome limits and barriers to its ease of use would be valuable.

### SUMMARY

The present disclosure is directed to systems and methods for interpreting and labeling medical ultrasound images. Various anatomical structures may be identified within the ultrasound image by matching the ultrasound images obtained by a user with images of similar views stored in a database. The ultrasound images collected by the user are displayed on a monitor screen in real-time. The system may apply labels of various types (e.g., different shapes, shades, sizes, transparencies, colors, types of outlining, etc.) that may be easy to read and provide quick interpretation of the anatomical structures shown in the ultrasound image. Further, the labels may help facilitate reliable identification of normal and abnormal anatomical structures.

Ultrasound exams are typically performed by moving the ultrasound probe back and forth, or by fanning or rotating the probe in multiple planes. Consequently, the ultrasound images are viewed in real-time in video format on the monitor screen. Typically, the ultrasound device has the ability to freeze the screen to look at areas of interest. The systems and methods disclosed herein may provide labeling of the ultrasound images as the anatomical structures appear on the monitor screen during the real-time video, or at least after a frame of the video is frozen on the monitor screen. The labels may appear automatically and disappear automatically based on various factors such as, for example, a particular size, shape, percentage portion, or clarity of an ultrasound image of a particular anatomical structure that is included in the ultrasound image. Images with and without the labels applied may be frozen on the screen and saved, stored, printed, or transmitted as desired.

Certain types of studies may benefit from different ultrasound modes such as, for example, colored Doppler or movement mode (M-mode). The system may have the ability to recognize a given study and switch to a mode that is most helpful for interpretation by the user and/or labeling by the system.

A further aspect of the present disclosure relates to a method of labeling medical ultrasound images that includes selecting a type of medical ultrasound study, selecting a probe orientation for the ultrasound study, obtaining ultrasound images from a patient using a probe positioned at the selected probe orientation, displaying the ultrasound images, comparing the ultrasound images to a database of known ultrasound images within the selected study and probe orientation, and automatically labeling anatomical structures shown in the ultrasound images.

The labeling may include shading at least some of the anatomical structures, outlining at least some of the anatomical structures, or adding at least one text label. The labeling may appear and disappear as the anatomical structures move into and out of view in the ultrasound images. The labeling may include displaying a list of labeling options and selecting at least one labeling option. The method may include altering an opacity feature of the shading. The method may include, after selecting the type of study, displaying a menu of anticipated landmark anatomical structures to appear in the ultrasound images, and selecting which landmark structure to automatically label. The method may include, after selecting the type of study, selecting from a plurality of ultrasonic views. The method may include displaying a plurality of probe orientation diagrams and selecting among the displayed probe orientation diagrams. The ultrasound images may comprise freeze frames of real-time ultrasound video.

Another aspect of the present disclosure relates to a method of identifying and labeling medical ultrasound images that includes providing an ultrasound probe, a display screen, a labeling module, and a database of ultrasound images, collecting ultrasound images of anatomical structures from a patient with the ultrasound probe, displaying the ultrasound images on the display screen, referencing the database of ultrasound images to identify anatomical structures within the ultrasound images using the labeling module, and labeling the anatomical structures displayed on the display screen with the labeling module.

The method may include selecting a type of medical ultrasound study from a plurality of medical ultrasound studies displayed on the display screen. The method may include selecting a probe orientation for the selected medical ultrasound study from a plurality of probe orientations displayed on the display screen. The labeling may include shading at least some of the anatomical structures, outlining at least some of the anatomical structures, or providing a text label for at least some of the anatomical structures. The method may include automatically adding and removing the labeling as the anatomical structures move into and out of view on the display screen.

A further example method accordance with the present disclosure relates to a method of labeling medical ultrasound images in real-time. The method includes obtaining real-time ultrasound video of a patient, taking ultrasound images from at least one frame of the ultrasound video, displaying the ultrasound images, comparing the ultrasound images to a database of known ultrasound images within the selected study and probe orientation, and automatically labeling anatomical structures shown in the ultrasound images.

The automatic labeling may include shading at least some of the anatomical structures, outlining at least some of the anatomical structures, or adding at least one text label. The automatic labeling may provide at least one label that appears and disappears as the anatomical structures move into and out view. The automatic labeling may include displaying a list of labeling options and selecting at least one labeling option.

The method may include altering an opacity feature of the shading. The method may include, after selecting the type of study, displaying a menu of anticipated landmark anatomical structures to appear in the ultrasound images, and selecting which landmark structure to automatically label. The method may include, after selecting the type of study, selecting from a plurality of ultrasonic views. The method may include displaying a plurality of probe orientations and selecting among the displayed probe orientations. The method may include selecting a type of medical ultrasound study prior to obtaining the real-time ultrasound video.

A further aspect of the present disclosure relates to a system for labeling medical ultrasound images. The system includes a processor, an ultrasound probe, a display screen, an ultrasound image database, and a labeling module. The display screen is configured to display ultrasound images collected by the ultrasound probe. The ultrasound image database includes a plurality of stored ultrasound images. The labeling module is configured to compare the ultrasound images displayed on the display screen to the stored ultrasound images and automatically label the ultrasound images displayed on the display screen.

The labeling module may display a list of medical ultrasound studies on the display screen for selection by a user. The labeling module may display a list of ultrasound probe orientations on the display screen for selection by a user. The list of ultrasound probe orientations may include diagrams for each ultrasound probe orientation. The automatic labeling by the labeling module may include shading at least some of the anatomical structures, outlining at least some of the anatomical structures, or providing a text label for at least some of the anatomical structures. The ultrasound images may be still shots taken from a real-time ultrasound video.

Features from any of the above-mentioned embodiments may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE DRAWINGS

The accompanying drawings illustrate a number of exemplary embodiments and are a part of the specification. Together with the following description, these drawings demonstrate and explain various principles of the instant disclosure.

FIG. 1 is a block diagram showing an example system in accordance with the present disclosure.

FIG. 2 is a block diagram showing another example system in accordance with the present disclosure.

FIG. 3 is a block diagram showing another example system in accordance with the present disclosure.

FIG. 4 is a block diagram showing an example labeling module in accordance with the present disclosure.

FIG. 5 is a flow diagram showing an example method in accordance with the present disclosure.

FIG. 6 is a flow diagram showing another example method in accordance with the present disclosure.

FIG. 7 is a flow diagram showing another example method in accordance with the present disclosure.

FIGS. 8-13 show labeling options for a standard heopatoreanal view of liver and kidney with abnormal free fluid in accordance with the present disclosure.

FIGS. 14-18 shown labeling options for a short axis ultrasound view of an aorta with abdominal aortic aneurysm in accordance with the present disclosure.

FIGS. 19-21 shown labeling options for a subxiphoid view of a pericardial effusion having pericardial fluid in accordance with the present disclosure.

FIGS. 22-24 shown labeling options for a neck ultrasound of an internal jugular vein and carotid artery in accordance with the present disclosure.

FIGS. 25-30 shown labeling options for a lung ultrasound in movement mode in accordance with the present disclosure.

FIG. 31 depicts a block diagram of a computer system suitable for implementing aspects of the present systems and methods.

FIG. 32 is a block diagram depicting a network architecture in which client systems, as well as storage servers (any of which can be implemented using computer system), are coupled to a network.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION OF THE DRAWINGS

One aspect of the present disclosure is directed to a computerized image recognition system and related methods in which anatomical structures are identified within an ultrasound image. Another aspect to the present disclosure relates to a computerized image labeling system and related methods wherein ultrasound images obtained in real-time are compared to a stored database of ultrasound images showing normal and abnormal anatomical structures. The system may provide quick and accurate matching, identifying, evaluating, and labeling of normal and abnormal structures within the ultrasound images collected and evaluated. Some of the anatomical structures shown in the ultrasound images may not otherwise be recognizable or interpretable by the user without the image recognition and automatic labeling provided by the example systems and methods disclosed herein. In one example, when a particular type of study is selected in advance of collecting the ultrasound image, the system may recognize anatomical structures based on, for example, shape, size, relative echogenicity, pattern, presence or absence of blood flow, proximity to other anatomical structures in the field of view, or other properties.

In addition to recognizing, evaluating, and labeling various normal and abnormal anatomical structures within an ultrasound image, another aspect of the systems and methods disclosed herein relates to the ability to measure various features, identify abnormalities such as fluid, injury, stones, swelling, abnormal dilation, shrinkage or atrophy, abnormal masses, abnormal air, or inflammation adjacent to or within the anatomical structures in the ultrasound image. Another aspect of the example systems and methods disclosed herein relates to identifying the presence or absence of blood flow or clots within vessels or other anatomical structures. The term anatomical structure may refer generally to tissue, fluids, voids or pockets that are free of tissue or fluids, directionality of flow, or movement of a tissue or fluid.

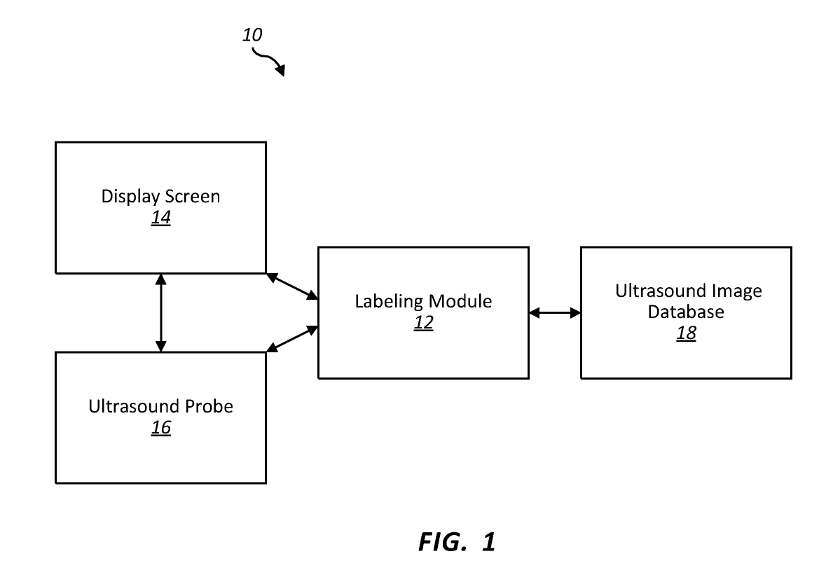

Referring now to FIG. 1, an example ultrasound image labeling system 10 is shown schematically including a labeling module 12, a display screen 14, an ultrasound probe 16, and an ultrasound image database 18. The ultrasound probe 16 may be used to collect a plurality of ultrasound images of various anatomical structures of a patient. The ultrasound images may be displayed on the display screen 14. The labeling module 12 may operate to analyze the ultrasound images to identify the anatomical structure shown in the ultrasound images.

The labeling module 12 may reference the ultrasound image database 18 to compare the ultrasound images to a plurality of stored ultrasound images in the ultrasound image database 18. After identifying the anatomical structures in the ultrasound image, the labeling module 12 may label the ultrasound images and display the labeling on the display screen 14.

In some arrangements, there is no direct communication between the ultrasound probe 16 and display screen 14. The ultrasound images provided by the ultrasound probe 16 may be routed through the labeling module 12 for analysis, including identification of anatomical structures in the ultrasound image and labeling of the ultrasound images prior to displaying the ultrasound images on the display screen 14.

The system 10 may include capability to select an ultrasound study prior to collecting ultrasound images with the ultrasound probe 16. An ultrasound study may be generally defined as an analysis or study of a particular area of the human body using an ultrasound probe. Selecting the ultrasound study may include selecting among a plurality of probe orientations or ultrasound views for the ultrasound probe 16. The probe orientation may include a position or orientation of the ultrasound probe 16 relative to a reference point on the human body. Selection of the ultrasound study may also include selection of a mode such as, for example, a movement mode (M-mode). The selection of probe orientation and mode may be provided as separate operational steps in using the system 10 prior to collecting ultrasound images with the ultrasound probe 16.

The labeling module 12 may use the selected ultrasound study, probe orientation, and mode to select among various categories of ultrasound images stored in the ultrasound image database 18. Preselection of the ultrasound study, probe orientation and mode may assist in accelerating the rate at which the labeling module 12 can identify anatomical structures and label the anatomical structures in the ultrasound images collected by ultrasound probe 16.

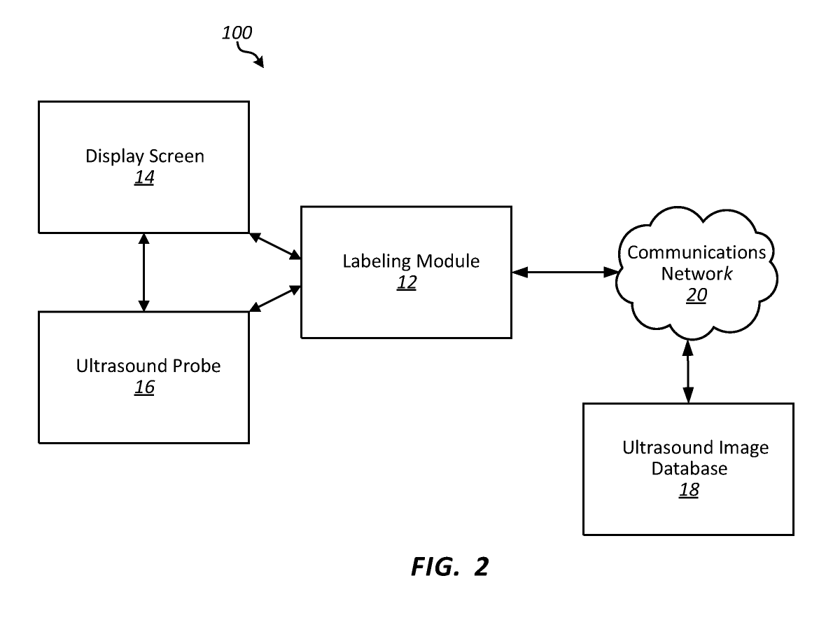

Referring now to FIG. 2, another example ultrasound image labeling system 100 is shown including the labeling module 12, display screen 14, ultrasound probe 16 and ultrasound image database 18 of system 10. System 100 further includes a communications network 20 through which the labeling module 12 accesses the ultrasound image database 18. In some arrangements, the ultrasound image database 18 is provided locally such as stored on a hard drive of a computer system that is operating the labeling module 12. In other examples, such as system 100, the ultrasound image database 18 is stored remotely and accessed via a communications network 20 such as, for example, the Internet, a local area network (LAN), or a wide area network (WAN). In still further arrangements, any of the labeling module 12, display screen 14, and ultrasound probe 16 may be positioned remote from each other as well. For example, the display screen 14 may be positioned remotely to provide visualization by a remotely located doctor who is attempting to diagnose a patient remotely. In other arrangements, the labeling module 12 may be stored and operated remote from the ultrasound probe 16 and/or the display screen 14. With the use of computers and various communications networks, the relative location of the various components of system 100 may be less relevant.

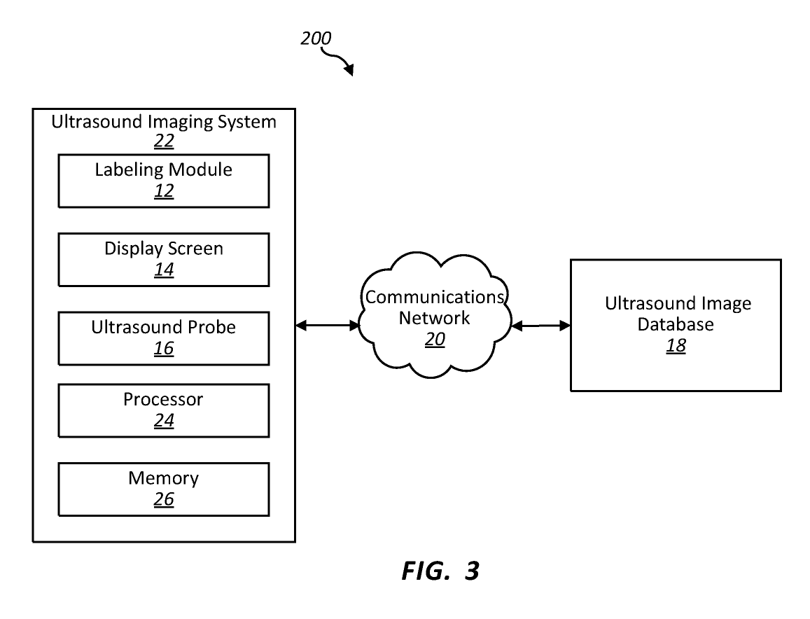

Referring to FIG. 3, an ultrasound image labeling system 200 includes an ultrasound imaging system 22 having the labeling module 12, display screen 14 and ultrasound probe 16 of the systems 10, 100 described above. Ultrasound imaging system 22 also includes a processor 24 and memory 26. The ultrasound imaging system 22 communicates with the ultrasound image database 18 via the communications network 20. The processor 24 and memory 26 may be useful in operating at least some of the labeling module 12, display screen 14 and ultrasound probe 16. In at least some arrangements, any one of the labeling module 12, display screen 14 and ultrasound probe 16 may include their own separate and distinct processor 24 and memory 26 rather than having a single processor 24 and memory 26 used to operate the entire ultrasound imaging system 22.

In at least some examples, the ultrasound imaging system 22 is configured as a stand alone system or assembly that is housed together and portable as a single unit or device. In one example application, the ultrasound imaging system 22 is housed on a mobile cart (or even within a handheld device) that is movable into and out of emergency room evaluation bays or other exam settings and used for quick diagnosis of a patient in an emergency medical situation. The ultrasound image database 18 may be accessible by the ultrasound imaging system 22 via the Internet or other communications network 20. In still further arrangements, the ultrasound image database 18 is included within the ultrasound imaging system 22 so as to be mobile and movable with the other components of the ultrasound imaging system 22.

The ultrasound image database 18 described with reference to systems 10, 100, 200 may be readily updated with additional images for a given ultrasound study, and may be updated to include ultrasound images for additional studies. The updates to ultrasound image database 18 may occur by downloading via, for example, the communications network 20. Alternatively, the updates are provided on a stored hardware device such as a CD-ROM or DVD-ROM or flash drive. As discussed above, the images stored on ultrasound image database 18 may be categorized by ultrasound study, ultrasound probe orientation, and ultrasound mode. There are numerous ways to organize and access ultrasound images stored in the ultrasound image database 18 as will be well known to those of skill in the art.

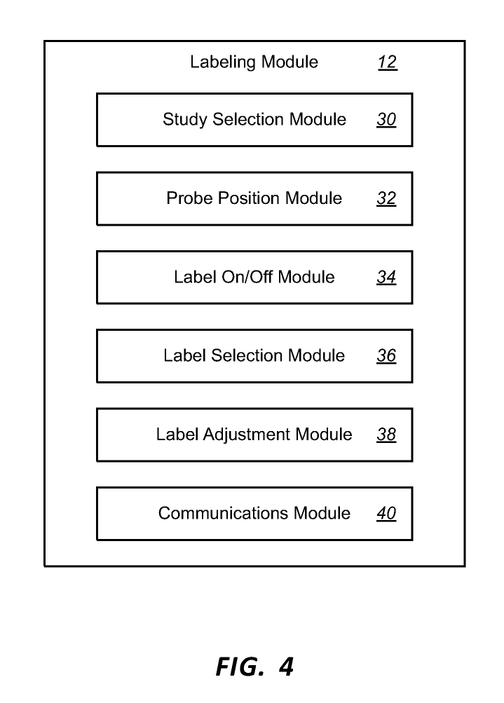

Referring now to FIG. 4, an example labeling module 12 is shown including a study selection module 30, a probe position module 32, a label on/off module 34, a label selection module 36, a label adjustment module 38, and a communications module 40. The various modules of the labeling module 12 are exemplary only and can be added to or removed from in order to provide a functional labeling module that performs the various functions and operations described herein.

The study selection module 30 may operate to provide a list of potential ultrasound studies for the user to select among. The list of potential ultrasound studies may be presented on the display screen 14. A list of potential ultrasound studies is provided below. Alternatively, the list of ultrasound studies to select among may be included or listed on a keypad wherein selection is made by pressing a button, clicking a mouse or keying in a code.

The probe position module 32 may provide at least one probe position option for each ultrasound study. Typically, once an ultrasound study is selected via the study selection mode 30, at least one probe position option may be presented to the user, for example, on the display screen 14. The various probe positions may include diagrams showing the user how the probe is oriented and where it is located relative to reference points on the patient's body. In other arrangements, the probe positions are merely listed or described with written text or an audible explanation. The selected probe position and its description or associated diagram may remain visible on the display screen 14 for the user to reference while collecting ultrasound images with the ultrasound probe 16.

The label on/off module 34 may include an on/off capability for whatever labeling is provided on the ultrasound image using the labeling module 12. In some arrangements, the labeling applied to the ultrasound images and displayed on display screen 14 may obstruct the user's view of at least portions of the anatomical structures in the ultrasound image. The labeling may be applied automatically with the labeling module 12 and the user may selectively turn the labeling on and off in order to obtain better or clearer visualization of the anatomical structures in the ultrasound image.

The label selection module 36 may present a plurality of label options to the user. The label options may be shown on display screen 14 or may be included on a keyboard or other device such as the ultrasound probe 16. Several example labeling options are described in further detail below. A plurality of different labels may be included on a single ultrasound image. Particular types of probe positions or ultrasound studies may have a list of labeling options that are automatically presented to the user. Alternatively, the user may manually select a custom set of labels for a particular ultrasound image. The labeling module 12 may have a default labeling that is applied to all ultrasound images.

The label adjustment module 38 may provide adjustment or customization of any one of the labels selected with the label selection module 36 or provided automatically as a default labeling to the ultrasound images. For example, the label adjustment module 38 may provide for adjusting an opacity of the label, adjusting a color of the label, adjusting a language of letters or descriptive terms of the labels or adjusting a timing by which the labels automatically appear and disappear within a real-time ultrasound video being collected by the ultrasound probe 16.

The labeling module 12 may provide communication with a user via the communications module 40. The communications may include, for example, suggestions or practical tips related to a selected ultrasound study, an explanation of an error, a listing of various potential diagnoses associated with a selected ultrasound study, warnings, etc. The communications module 40 may provide communication via, for example, the display screen 14 or ultrasound probe 16. In some embodiments, the communications module 40 may provide communication via an audible signal or audible description.

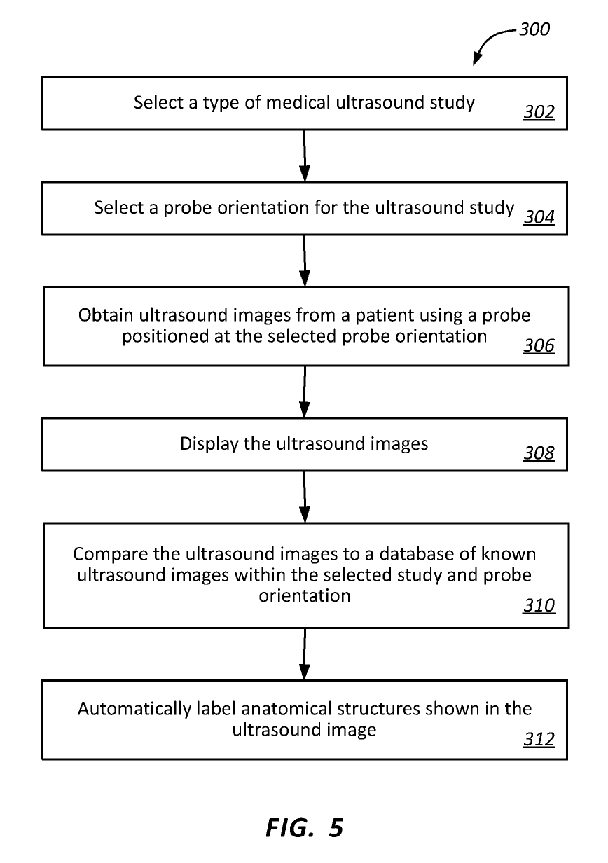

Referring to FIG. 5, an example method 300 of labeling a medical ultrasound image includes a first step 302 of selecting a type of medical ultrasound study. A further step 304 includes selecting a probe orientation for the ultrasound study. An ultrasound image is obtained from a patient using a probe positioned at the selected probe orientation in a step 306. A step 308 includes displaying the ultrasound image 308. The ultrasound images are compared to a database of known ultrasound images within the selected study and probe orientation in a step 310. A particular ultrasound study, for example a cardiac study, might have numerous views (e.g., apical, parasternal and subxiphoid). One or more of these views might have numerous standard probe orientations (e.g., parasternal long axis, or parasternal short axis). Other studies, for example, a soft tissue study looking for a foreign body or fluid collection, might use multiple unnamed or nonstandard views in multiple planes to localize various structures. The method further includes automatically labeling anatomical structures shown in the ultrasound image in a step 312.

Additional steps related to the method 300 may include shading at least some of the anatomical structures as a means of labeling. The labeling may include outlining at least some of the anatomical structures or adding at least one text label to the ultrasound image. The labeling may appear and disappear as the anatomical structures move into and out of view in the ultrasound images. The labeling may appear after a frame of the real-time ultrasound video is frozen. Labeling may include displaying a list of labeling options and selecting at least one labeling option.

The method 300 may also include the ability to adjust or alter the labeling such as, for example, altering an opacity feature of the shading label. The method 300 may include, after selecting the type of study, displaying a menu of anticipated landmark anatomical structures to appear in the ultrasound image, and selecting which landmark structure to automatically label. The method 300 may include displaying a plurality of probe orientation diagrams and selecting among the displayed probe orientation diagrams as part of the selection of probe orientation. The ultrasound images may be freeze frames of real-time ultrasound video.

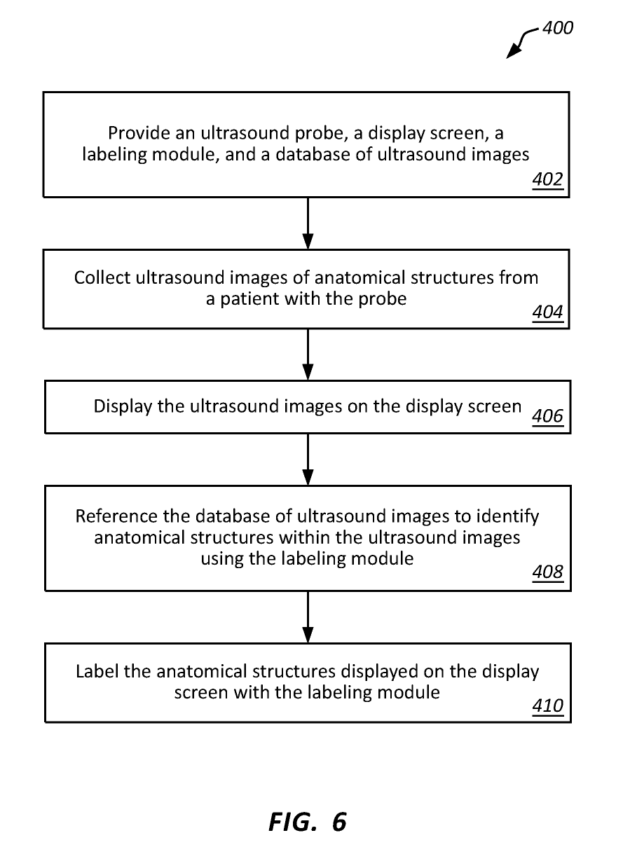

Referring now to FIG. 6, another example method 400 relates to identifying and labeling medical ultrasound images. The method 400 includes providing an ultrasound probe, display screen, a labeling module, and a database of ultrasound images in a step 402. A step 404 includes collecting ultrasound images of anatomical structures from a patient with the probe. The ultrasound images are displayed on the display screen in a step 406. The database of ultrasound images is referenced to identify anatomical structures within the ultrasound images using the labeling module in a step 408. A step 410 includes labeling the anatomical structures displayed on the display screen with the labeling module.

Other example steps related to method 400 may include selecting a type of medical ultrasound study from a plurality of medical ultrasound studies displayed on the display screen. The method 400 may include selecting a probe orientation for the selected medical ultrasound study from a plurality of probe orientations displayed on the display screen. Labeling may include shading at least some of anatomical structures, outlining at least some of the anatomical structures, or providing a text label for at least some of the anatomical structures. The method 400 may include automatically adding and removing the labeling as the anatomical structure is moved into and out of view on the display screen.

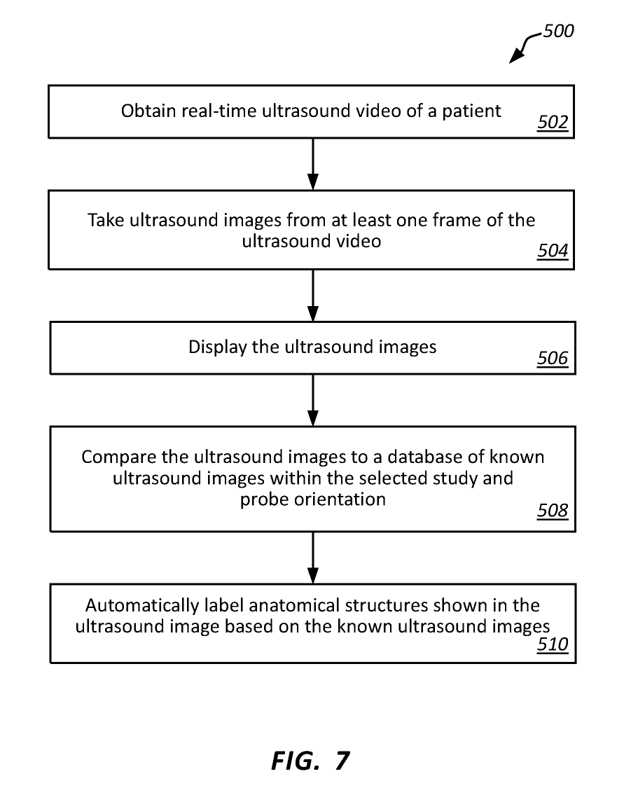

FIG. 7 shows another example method 500 related to labeling medical ultrasound images in real-time. The method 500 may include obtaining real-time ultrasound video of a patient in a step 502. A step 504 may include taking ultrasound images from at least one frame of the ultrasound video. A step 506 may include displaying the ultrasound images. The ultrasound images are compared to a database of known ultrasound images within the selected study and probe orientation in a step 508. The anatomical structures are automatically labeled based on the known ultrasound images in a step 510.

Other steps of the method 500 may include automatically labeling by shading at least some of the anatomical structures, outlining at least some of the anatomical structures, or adding text to label at least some of the anatomical structures. The automatically labeling step may provide at least one label that appears and disappears as the anatomical structures move into and out of view. The automatically labeling step may include displaying a list of labeling options and selecting at least one of the labeling options.

The method 500 may include altering an opacity feature of a labeling that includes shading. The method 500 may include, after selecting the type of study, displaying a menu of anticipated landmark anatomical structures to appear in the ultrasound images, and selecting which landmark structure to automatically label. The method 500 may include, after selecting the type of study, selecting from a plurality of ultrasonic views. The method may include displaying a plurality of probe orientations and selecting them on the displayed probe orientations. The method 500 may include selecting a type of medical ultrasound study.

Example medical ultrasound studies and potential uses for the systems and methods described herein include, for example and without limitation:
Trauma exams (FAST exam: Focused Assessment with Sonography for Trauma) to identify abnormal fluid or air
Evaluation of the aorta for abdominal aortic aneurysms, dilation of aortic root, dissection, acute occlusion
Evaluation of shock states (RUSH Exam: Rapid Ultrasound in Shock)
Evaluation of a hepatobiliary system for gallstones, gallbladder wall abnormalities, biliary inflammation and obstruction, pericholecystic fluid, common bile duct dilation
Echocardiogram for pericardial effusion and tamponade, cardiac activity and contractility, heart chamber size, thrombus, detection of central venous volume status, assessment of undifferentiated hypotension
Evaluation of the thorax to identify pneumothorax, pleural fluid or hemothorax (and to assist with drainage), pulmonary edema, pneumonia, inflammatory disorders, trauma
Identification of landmarks and vessels for central venous cannulation, peripheral vein cannulation, and arterial line cannulation
Diagnosis of deep vein thrombosis
Evaluation of deeper structures for localization of fluid collections or abscesses
Diagnosis peri-tonsillar abscess
Musculoskeletal and soft tissue exams (including evaluation of soft tissue infection for abscess, cellulitis, necrotizing fasciitis, identification of fractures, identification/removal of foreign bodies, evaluation of tears or injuries to muscles and tendons, identification of peripheral nerves for injuries and for anesthetic blocks, diagnosis of joint effusions or bursitis, evaluation of hip dysplasia, identification of landmarks for lumbar puncture
Abdominal ultrasound to identify peritoneal fluid, ascites or hemorrhage
Evaluation of bowel (obstruction, appendicitis, pyloric stenosis, diverticulitis)
Evaluation of pregnancy for detection of intrauterine pregnancy, ectopic pregnancy, detection of fetal heart rate, dating of pregnancy, detection of free fluid
Evaluation of urinary tract for kidney or ureter stones, hydronephrosis, bladder status
Recognition of enlarged organs
Pelvis ultrasound for ovarian torsion, cyst, mass, uterine abnormalities
Endoscopic ultrasound
Scrotal ultrasound for evaluation of its contents and blood flow
Evaluation of vasculature for dialysis catheters
Evaluation of blood flow in neck vasculature
Transcranial Doppler
Evaluation of intracranial hemorrhage in newborns through the fontanel
Identify esophageal versus tracheal intubations
Ocular ultrasound to assess retinal detachments, hemorrhage, dislocations or ruptures, posterior chamber pathology, optic nerve sheath diameter, or other abnormalities
Procedural guidance

When normal or abnormal anatomic structures or other features are recognized by the labeling module by reference to the ultrasound image database 18, the labeling module 12 may label the structures on the display screen 14. The type of label may depend on the particular study or anatomical structure. For example, some structures are too small to allow adequate room for a label to be positioned within the confines of the anatomical structures' image. Depending on the size and shape of the anatomical structure being evaluated, there may be multiple options for labeling the anatomical structure of interest shown in the ultrasound image. The following are some of the many types of labeling that may be possible.

### Shading

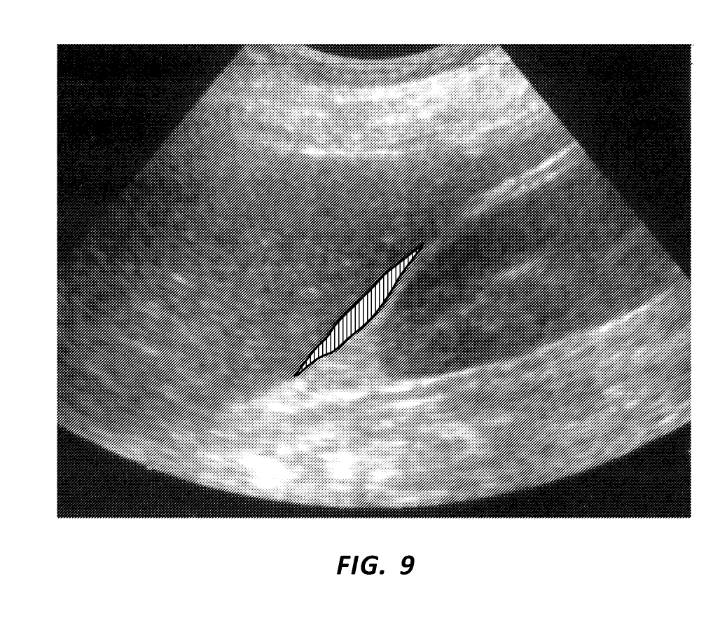

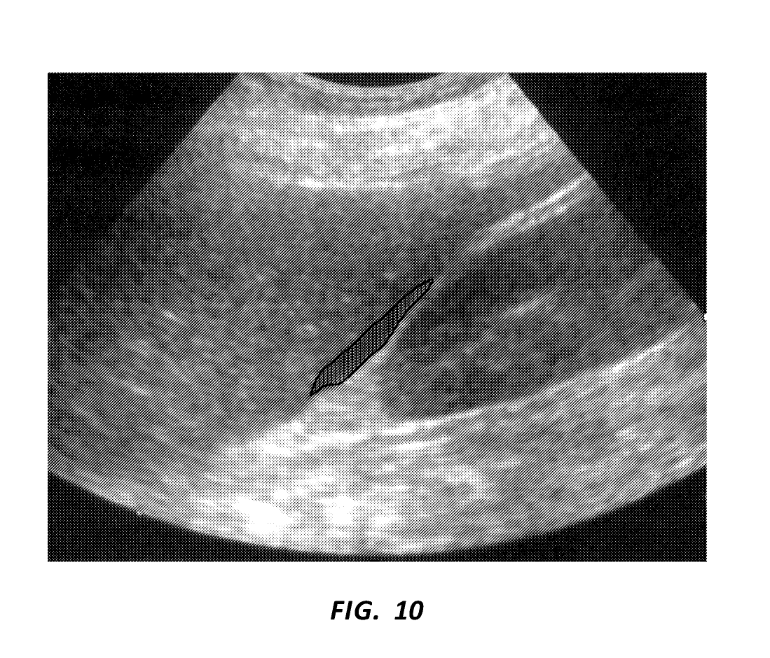

Shading added to the anatomical structures may over-lie the entire structure or a portion of the structure. The shading may be easily visible, variable in size and shape, and be provided with different colors. The shading may be transparent with controllable degrees of opacity that permit the user to simultaneously visualize details of the unlabeled image (e.g., see FIGS. 9 and 10).

### Lines

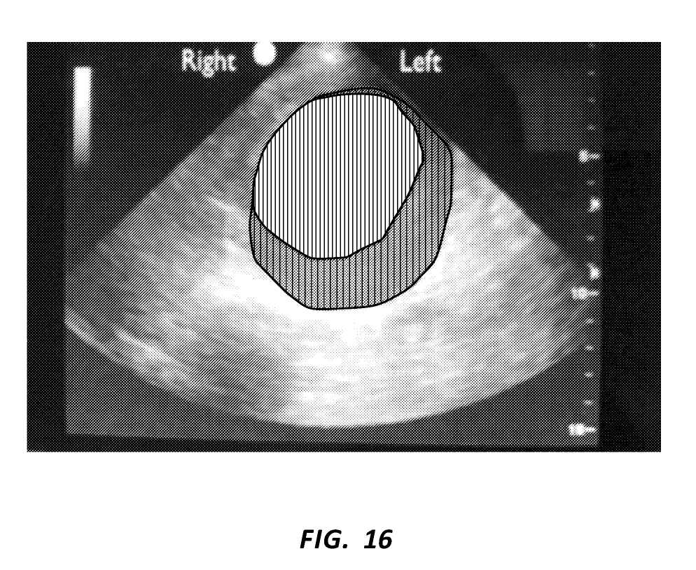

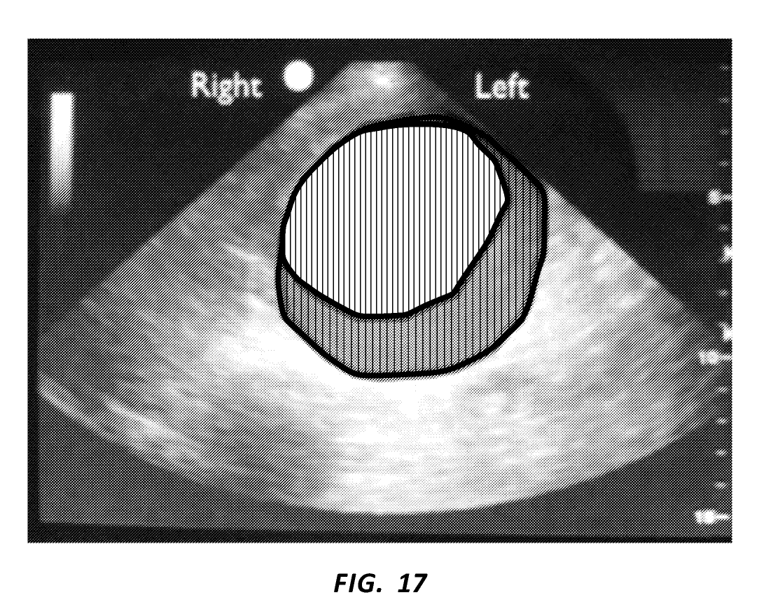

Lines may be used to delineate the edges of the anatomical structures of interest. The lines may be curved or straight, thick or thin, colored or black and white. FIGS. 16 and 17 show lines of various thickness.

### Text

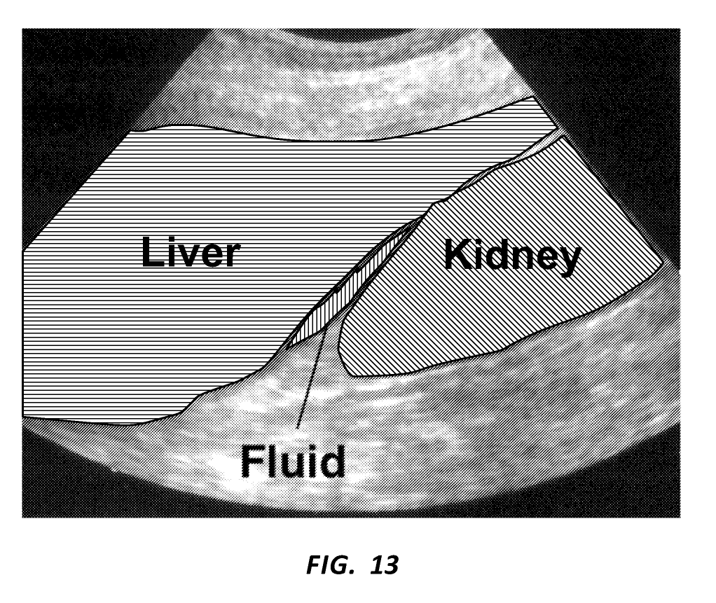

Text may be used to identify the anatomical structures or portions of the anatomical structures. The label may be contained within a boundary of the anatomical structure or have an arrow or line pointing from the text label to the anatomical structure depending on, for example the size, font, or other characteristic of the text or the size and shape of the anatomical structure. FIGS. 13, 18, 21 and 24 show various types of text labels. The text may be abbreviations or acronyms of full words such as names of a particular anatomical structure.

The type of label and various characteristics of the label may be set as a default for all ultrasound images collected by the system, or at least for particular types of ultrasound studies that have been selected. Alternatively, the user may opt to customize any one of the particular label options or a set of labeling options.

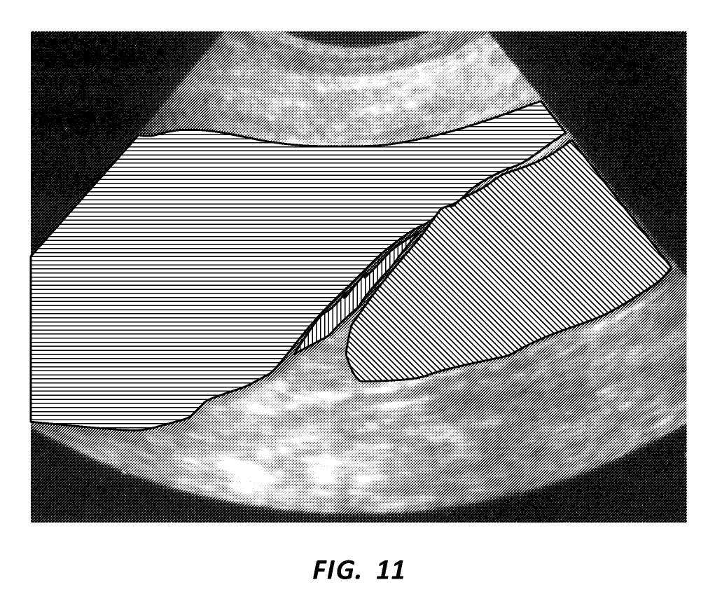

In one arrangement, different types of exams or ultrasound studies may have their own custom menu for choosing which anatomical structures will be labeled. For example, when evaluating Morrison's pouch for presence of abnormal fluid during a FAST exam (focused assessment with sonography for trauma), the user may prefer to have the liver and kidneys labeled with shading for orientation purposes (i.e., see FIGS. 11-13) or only have the abnormal fluid labeled (i.e., see FIGS. 9 and 10).

### Automatic Labeling

In practice, ultrasound exams are often performed by moving the ultrasound probe back and forth or by fanning or rotating a probe in different planes. This typically makes important anatomical structures come into and out of view during the examination. The labeling module 12 and related systems described herein may include the ability to have the labeling appear and disappear on the display screen 14 as the corresponding structure moves into and out of view. One advantage related to this feature is that small structures, fluid collections, or other findings, which may be missed when the anatomical structure is only briefly in view, will now have a colored appearance that provides a visual queue that is more easily seen on the display screen 14.

If while moving the ultrasound probe, the desired image or structure moves out of view, the user will know that he is no longer looking at an adequate ultrasound image because the label disappears. In addition, the ability to have labeling appear and disappear has the advantage of helping the user maintain orientation and proper probe position in order to obtain optimal images. The ultrasound images may be frozen on the screen, saved, printed and transferred with or without the labeling included on the ultrasound images.

### On/Off Functionality

While some expert users may argue that adding shading marks for labeling can interfere with the ability to visualize details of the original unlabeled ultrasound image, such labeling may be important for novice and inexperienced users to identify the anatomical structures in the ultrasound image. One option for permitting the user to have optimized visualization of all details of the unlabeled ultrasound image is to have the labeling be semi-transparent with an adjustable degree of opacity. This adjustability may allow the user to simultaneously visualize labels and subtle details of the original ultrasound image. In addition, the mode for visualizing shaded labels, markers, or other labeling may be turned on or turned off completely, if desired. Advanced users who prefer to use labels only for initial orientation and identification of anatomical structures may then have the option to remove all labeling or at least some labeling in order to allow unaltered views of the original ultrasound image.

### View Selection

As discussed above, the systems disclosed herein may prompt the user to select a type of ultrasound study such as an intended organ system, portion of a body limb, etc., in order for the system to properly recognize, interpret, and label the anatomical structures. The type of study may be selected in numerous ways such as, for example, on a touch screen from a list of potential ultrasound studies, using a keystroke, or with a mouse click from a list displayed on display screen 14. In at least one example, a list of all currently recognizable types of ultrasound studies may be available for selection and newly recognized ultrasound studies may be added as desired. In some arrangements, a limited number of ultrasound studies, or groups of studies, may be available depending on the environment or application for the system (e.g., an emergency room setting versus an orthopedic clinic).

After entering or selecting the type of ultrasound study, the user may be prompted to select from a list of standard views for that particular study. The menu of standard views may include diagrams or figures indicating a preferred probe type and probe orientation. This step may have the distinct advantage of reminding the inexperience or infrequent user where to place and how to position a probe for the best chance of obtaining optimized ultrasound images. Normal ultrasound images (i.e., those images expected from the same or very similar views), with or without labeling, may be available to look at on the monitor for orientation purposes. The reference or example image may remain on the monitor screen, (e.g., occupying a small corner at the top of the screen) for a visual example of an ideal image.

### Customized Labeling

When the particular view to be obtained via a particular probe orientation/position and study is selected, labeling options for the expected image (i.e., for both normal and abnormal anatomical structures) may be made available for the user. The user may select which anatomical structures to label. The user may also select among various options for the labeling such as, for example, colors, degree of opacity, text, border thickness, positioning of labeling, etc. The user may also select among specific abnormalities that may be of particular interest or that may be expected or suspected, and these abnormalities can be highlighted or receive a desired type of labeling (e.g., color, text, etc.). Custom preferences may be inputted, although the default settings may be intended to maximize clarity of the labeling process for a particular study or a probe orientation/view.

### Mode Selection

When determining the presence or absence of blood flow or tissue movement, (e.g., evaluating blood vessels, differentiating blood vessels from nerves or other structures, documenting blood flow to organs such as ovaries, or for evaluating movement of structures), colored Doppler or movement mode (M-mode) may be used. With current technology, selection of mode may be done manually by pushing a button or selecting an option on a touch screen. One aspect of the systems disclosed herein may be that the labeling module 12 or other aspect of the systems selects and changes modes if the selected study requires or is optimized using a different mode to assist with interpretation and labeling of the ultrasound images. Certain studies may benefit from different modes. The system may have the ability to recognize the studies and switch modes automatically as needed, or prompt the user to select a different mode manually.

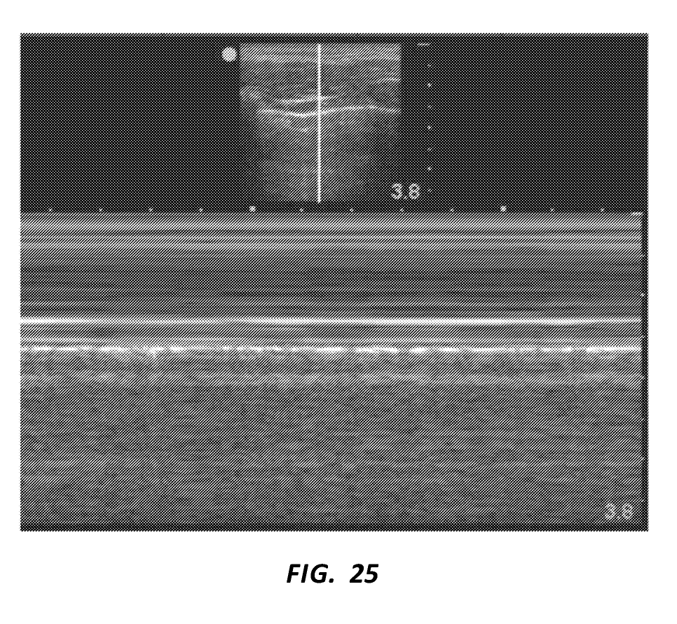

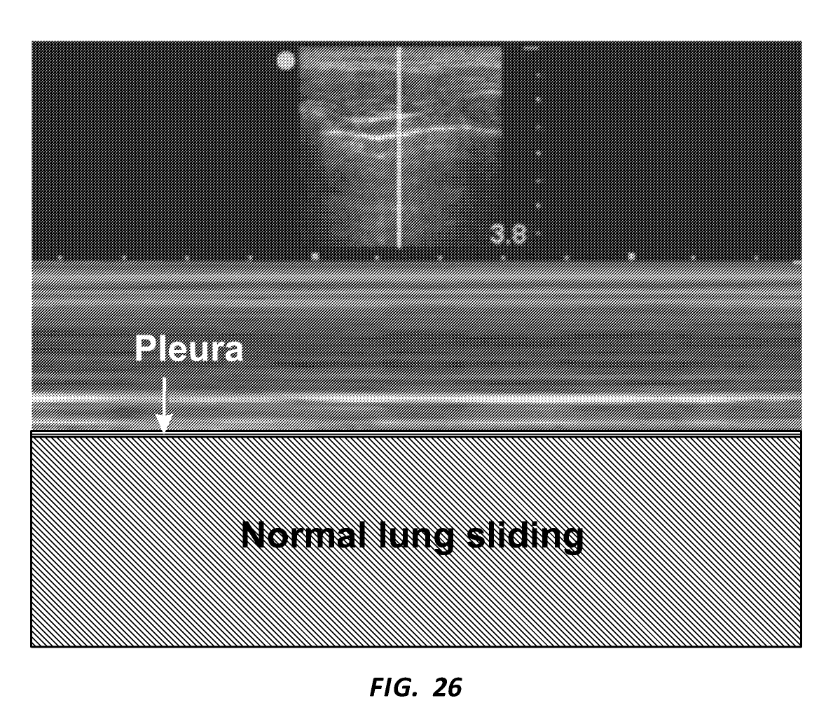

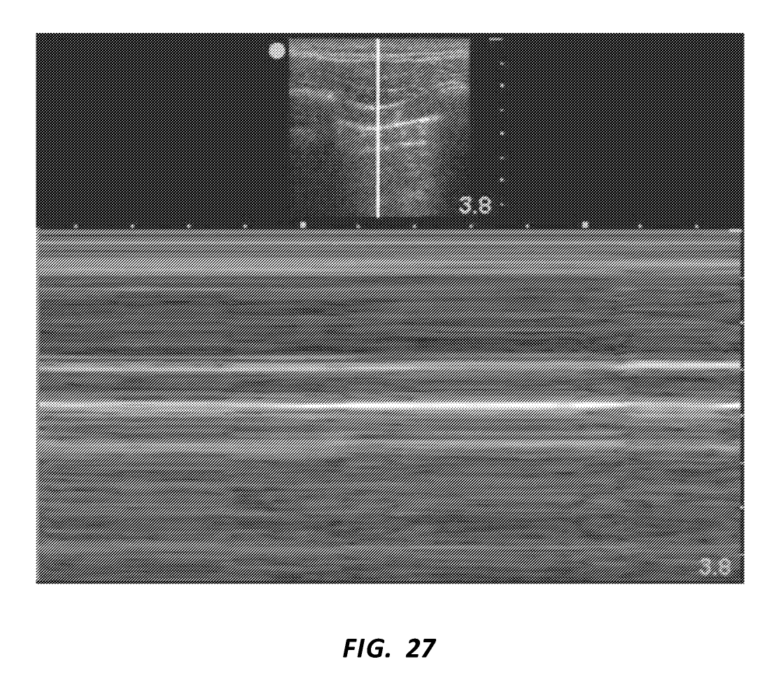

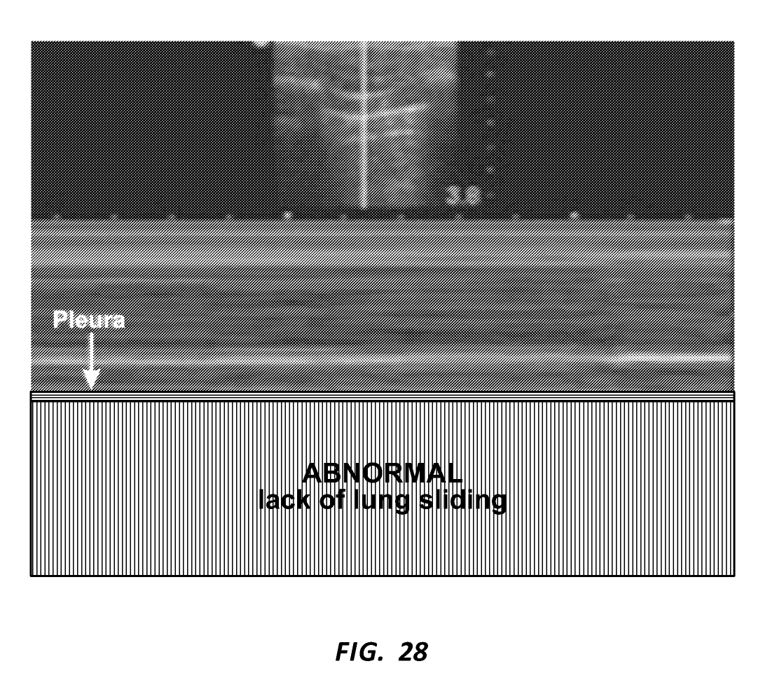

FIGS. 25-30 show images of a lung ultrasound using M-mode to detect pneumothorax. The abnormal lack of lung sliding or stratosphere sign seen with pneumothorax (see FIGS. 27 and 28) is more easily differentiated from normal lung sliding (see FIGS. 25 and 26) with labels on M-mode.

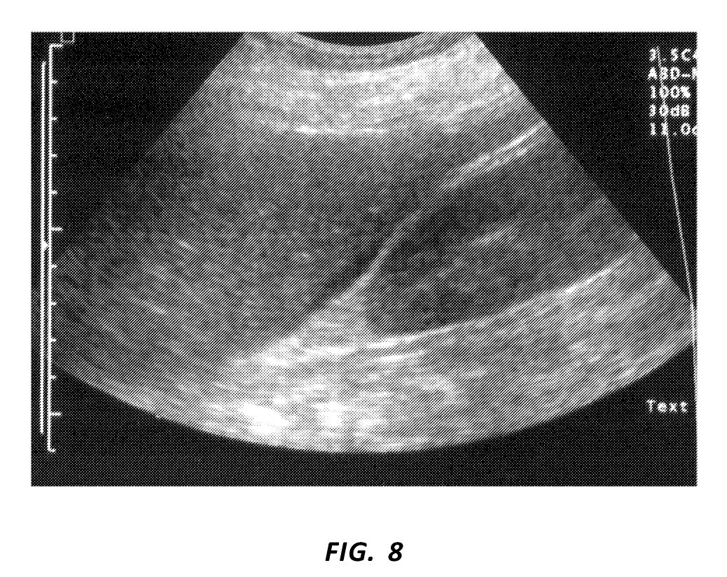

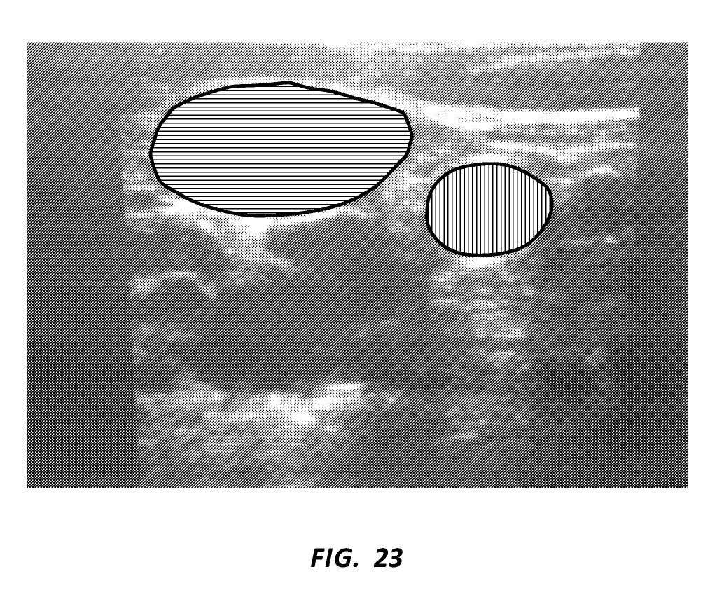

Further detail is provided related to the ultrasound images and associated labeling shown in FIGS. 8-30. FIGS. 8-13 show standard hepatorenal view of liver and kidney with abnormal free fluid in Morrison's pouch on FAST exam. FIG. 8 shows the ultrasound image with no labels or shading. Free fluid between the liver and kidney may be difficult to identify for an inexperienced user. FIG. 9 shows the abnormal free fluid between the liver and kidney labeled with the labeling module disclosed herein and shaded in red. FIG. 10 shows the labeling of the abnormal free fluid with an opacity decreased by about 30 to 40 percent. The opacity may be decreased further to allow a more transparent view of the rest of the ultrasound image, if desired.

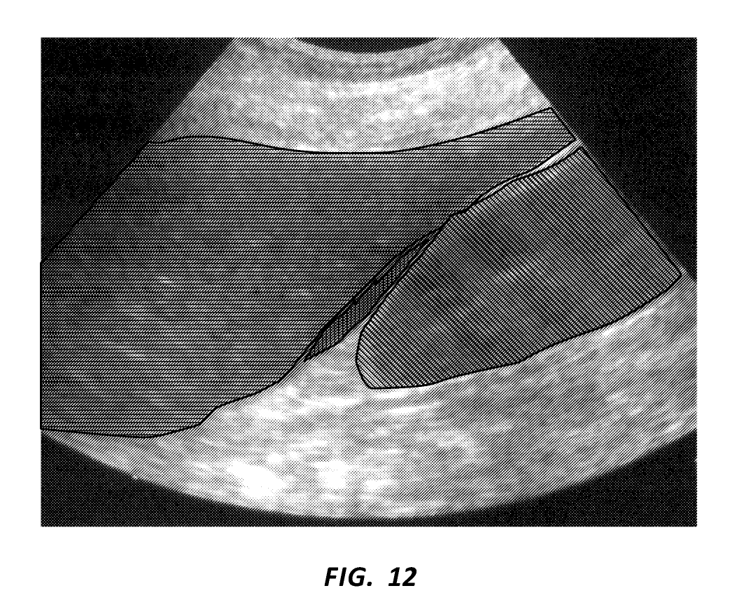

FIG. 11 shows the addition of shaded labels to assist with orientation of the abnormal free fluid by outlining other structures adjacent to the abnormal free fluid such as the liver (blue shading on the left) and kidney (green shading on the right). FIG. 12 shows the labels of FIG. 11 with a decreased opacity. The opacity of the shading may be further decreased to allow a more transparent view of the rest of the ultrasound image, if desired. FIG. 13 shows the addition of text labeling wherein the text labeling is the name of the organs or other anatomical structure. The text labeling appears within the structure, (i.e., the liver and kidney) or adjacent to the structure with an arrow or line indicating location of the structure described (i.e., the fluid).

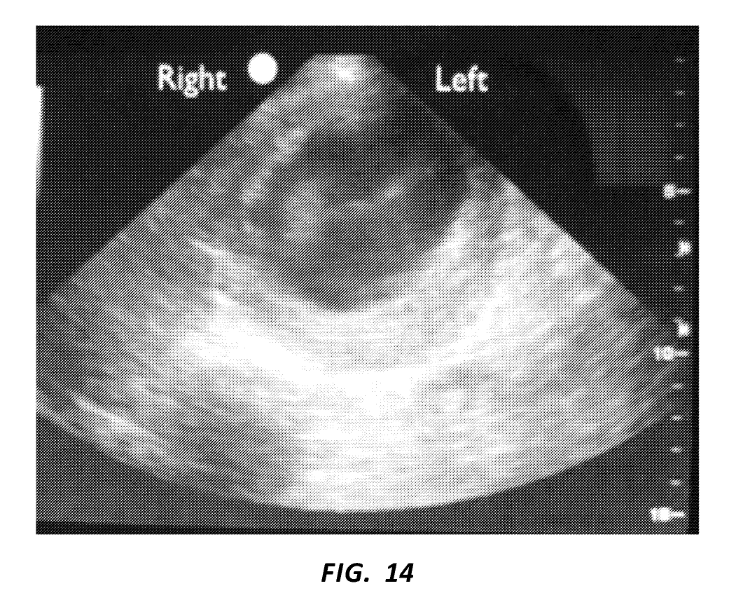

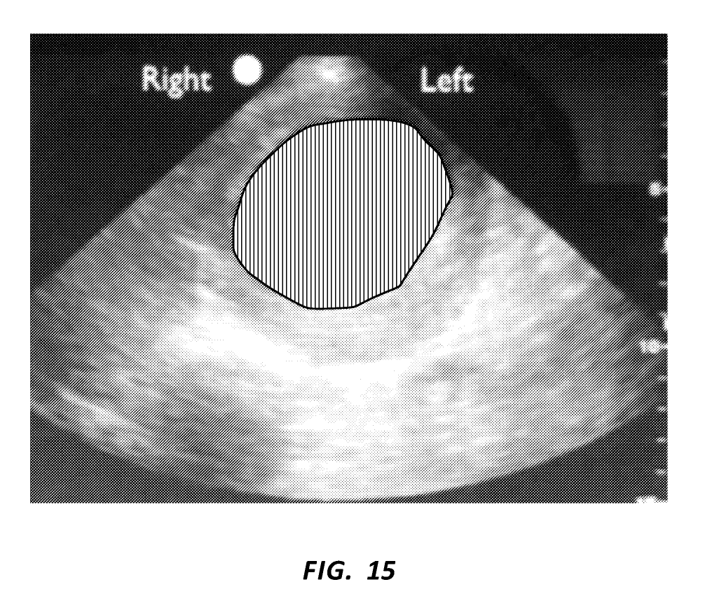

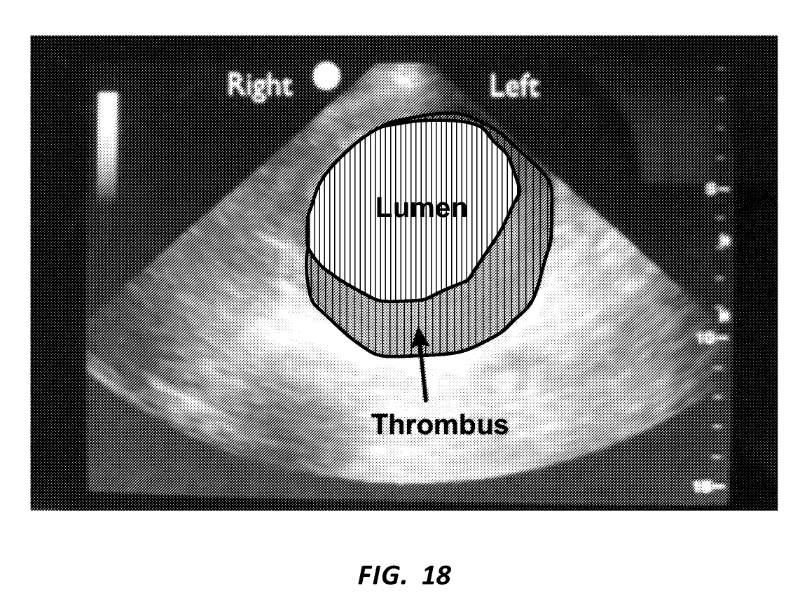

FIGS. 14-18 show a short axis ultrasound view of the aorta with abdominal aortic aneurysm. The aneurysm may be missed in FIG. 14 by an inexperienced user without the addition of labeling. FIG. 15 shows labeling added to the lumen of the aorta using the labeling system disclosed herein. The lumen of the aorta is shaded in red with a black outline. FIG. 16 shows the shading of both the aortic lumen and the aortic thrombosis with separate colors and thin lines outlining the areas of interest. The thrombosis is shaded with a pink shading. FIG. 17 shows the aortic lumen and thrombosis shaded with color and thicker outlines around the areas of interest. FIG. 18 includes the addition of text labeling to the color labeling shown in FIG. 16. Text labeling may be included overlapping the area of interest (i.e., the lumen) or positioned outside the anatomical structure or area of interest with a line pointing to the area of interest (i.e., the thrombosis).

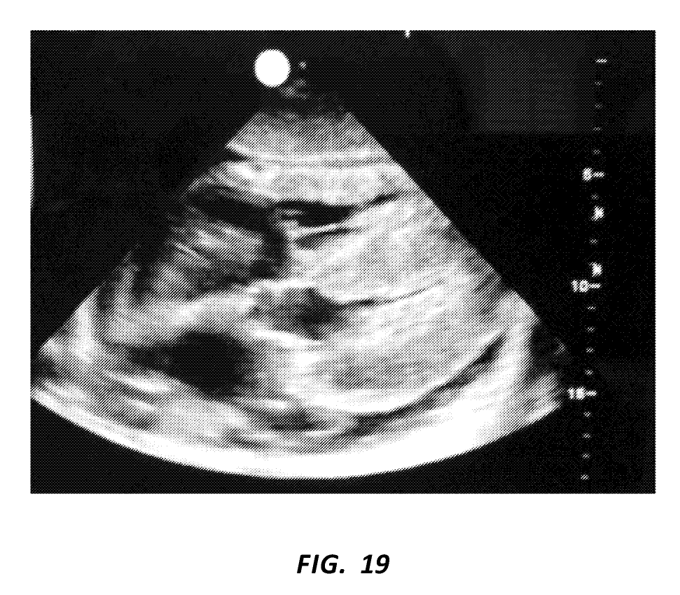

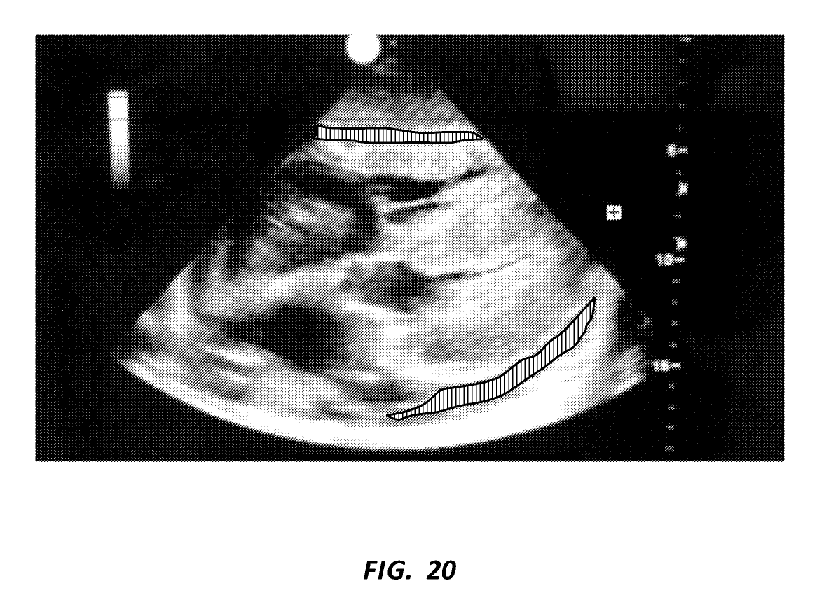

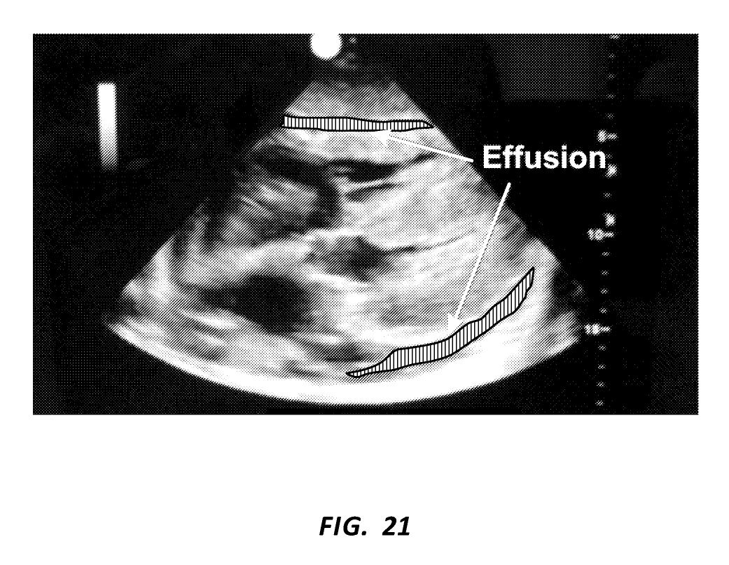

FIGS. 19-21 show a pericardial effusion, subxiphoid view, with no shading or labels included in FIG. 19. Without shading or labeling, the pericardial fluid may be easily missed by an inexperienced user. FIG. 20 shows the pericardial fluid highlighted with shading in red. FIG. 21 shows additional text labeling included pointing to the shaded abnormal fluid. The shading and text may appear and disappear or shrink and enlarge with the amount of fluid visible on the ultrasound image. In some examples, the opacity, thickness or intensity of outline or text may change as the abnormal fluid size increases.

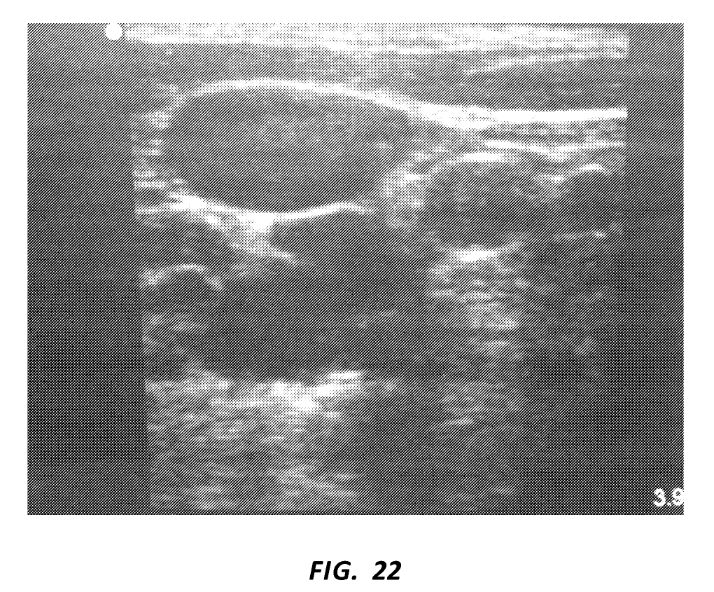

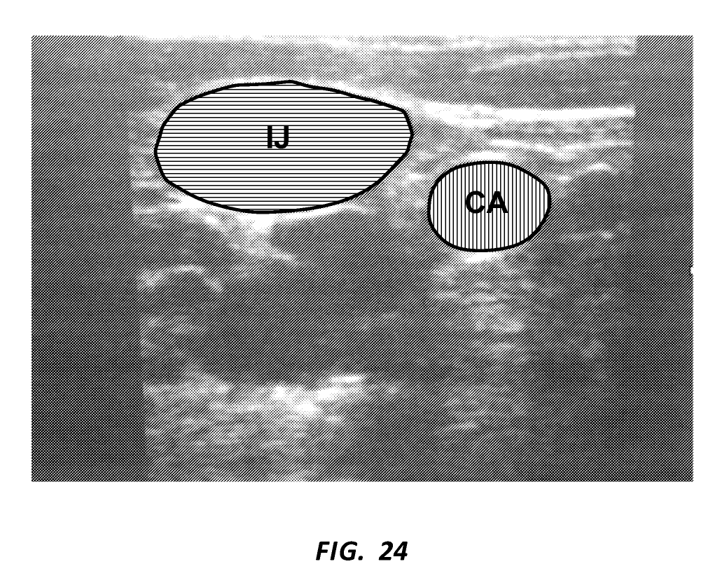

FIGS. 22-24 show a neck ultrasound with an internal jugular vein and carotid artery as the areas of interest. FIG. 22 does not include any labeling such as shading or text, making it difficult to identify which of the circular structures is the jugular vein versus the carotid artery. FIG. 22 shows the internal jugular vein shaded in blue (on the left) and the carotid artery shaded in red (on the right). FIG. 24 includes additional text labeling with the text provided as acronyms of the internal jugular vein and carotid artery. Either one of the labelings (i.e., shading or text) may appear automatically and may appear separately depending on certain characteristics of the anatomical structures shown in the ultrasound image. In one example, the shading may move or change shape with neck movement, with compression by the probe (i.e., normal venous compression), or with movement of the probe.

FIGS. 25-30 show various lung-related ultrasound images. FIG. 25 shows a normal lung sliding as seen in movement mode (M-mode). The normal grainy artifact is seen below the bright white pleural line as the pleural surfaces slide against one another. This feature may be important in excluding pneumothorax. With no labeling included in FIG. 25, it may be difficult for an inexperienced users to identify the features of interest. FIG. 26 includes labels with shading and text to identify the anatomical features of interest in the lung ultrasound image. The pleural line is labeled in blue as a thin line with the text label pleura in red pointing to the blue line. The normal lung sliding is shown with green shading at the bottom of the image with text explaining the normal lung sliding.

FIG. 27 is an ultrasound image of an abnormal lack of lung sliding in M-mode. The pleural appears stationery with hyperechoic lines visible during respiration. The “stratosphere sign” suggests lack of normal pleural movement and possible pneumothorax. Those relatively inexperienced in lung ultrasound may not be able to understand the various features shown in the ultrasound image of FIG. 27. FIG. 28 shows the abnormal lack of lung sliding labeled with red shading and text at the bottom of the image below the pleural line (labeled as blue with yellow text pointing to the blue line). The labeling may be turned on and off or changed in opacity in order for the user to see the image with or without the labeling once the labeling helps the user orient the features of interest.

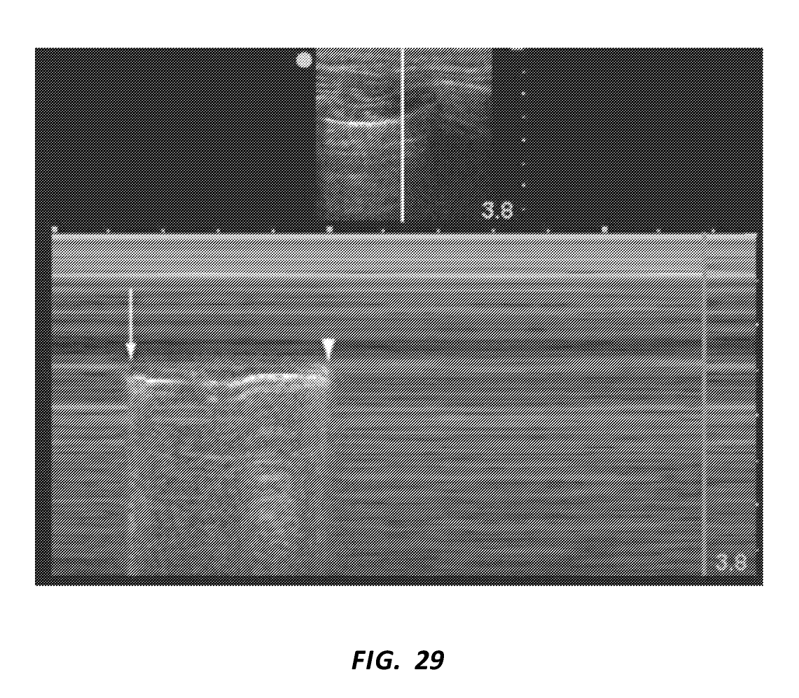

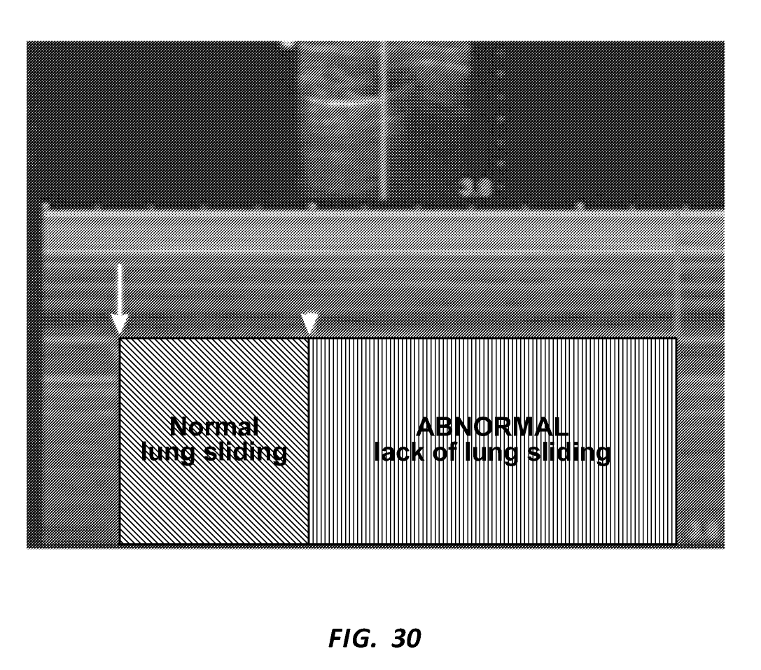

FIG. 29 shows a “lung point” sign in M-mode that is positioned at the interface between normal lung sliding and absence of lung sliding in a single view. This comparison in a single view may suggest to the user that pneumothorax is present. FIG. 30 includes labeling of the normal lung sliding portion of the image and the abnormal lack of lung sliding portion in the image. The labeling includes shading of green for the normal lung sliding on the left and red for the abnormal lack of lung sliding on the right.

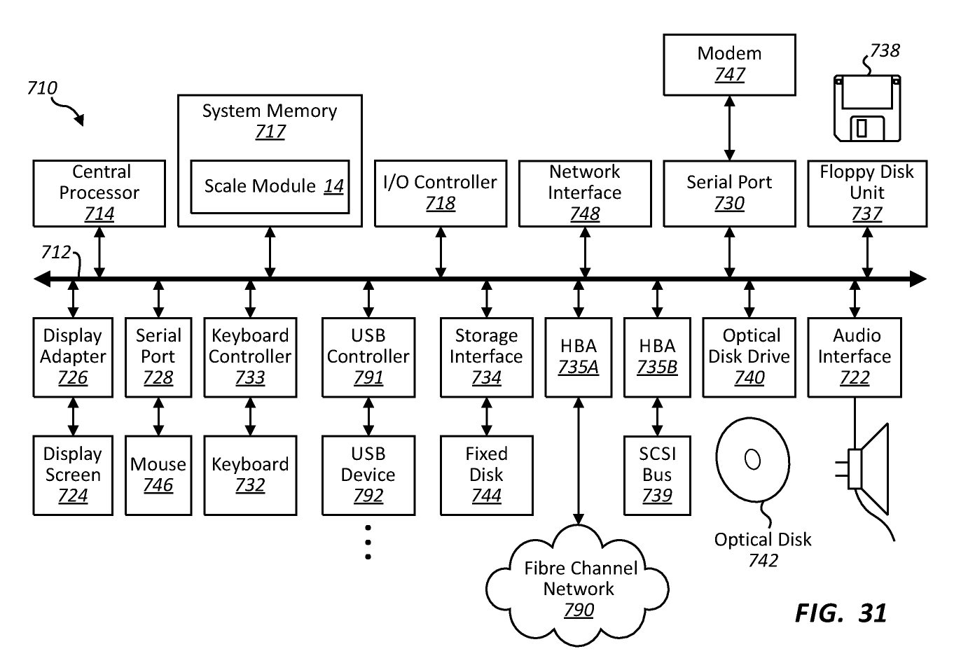

FIG. 31 depicts a block diagram of a computer system 710 suitable for implementing various aspects of the present systems and methods. Computer system 710 includes a bus 712 which interconnects major subsystems of computer system 710, such as a central processor 714, a system memory 717 (typically RAM, but which may also include ROM, flash RAM, or the like), an input/output controller 718, an external audio device, such as a speaker system 720 via an audio output interface 722, an external device, such as a display screen 724 via display adapter 726, serial ports 728 and 730, a keyboard 732 (interfaced with a keyboard controller 733), multiple USB devices 792 (interfaced with a USB controller 790), a storage interface 734, a floppy disk drive 737 operative to receive a floppy disk 738, a host bus adapter (HBA) interface card 735A operative to connect with a Fibre Channel network 790, a host bus adapter (HBA) interface card 735B operative to connect to a SCSI bus 739, and an optical disk drive 740 operative to receive an optical disk 742. Also included are a mouse 746 (or other point-and-click device, coupled to bus 712 via serial port 728), a modem 747 (coupled to bus 712 via serial port 730), and a network interface 748 (coupled directly to bus 712).

Bus 712 allows data communication between central processor 714 and system memory 717, which may include read-only memory (ROM) or flash memory (neither shown), and random access memory (RAM) (not shown), as previously noted. The RAM is generally the main memory into which the operating system and application programs are loaded. The ROM or flash memory can contain, among other code, the Basic Input-Output system (BIOS) which controls basic hardware operation such as the interaction with peripheral components or devices. For example, the labeling module 12 to implement the present systems and methods may be stored within the system memory 717. Applications resident with computer system 710 are generally stored on and accessed via a computer readable medium, such as a hard disk drive (e.g., fixed disk 744), an optical drive (e.g., optical drive 740), a floppy disk unit 737, or other storage medium. Additionally, applications can be in the form of electronic signals modulated in accordance with the application and data communication technology when accessed via network modem 747 or interface 748.

Storage interface 734, as with the other storage interfaces of computer system 710, can connect to a standard computer readable medium for storage and/or retrieval of information, such as a fixed disk drive 744. Fixed disk drive 744 may be a part of computer system 710 or may be separate and accessed through other interface systems. Modem 747 may provide a direct connection to a remote server via a telephone link or to the Internet via an interne service provider (ISP). Network interface 748 may provide a direct connection to a remote server via a direct network link to the Internet via a POP (point of presence). Network interface 748 may provide such connection using wireless techniques, including digital cellular telephone connection, Cellular Digital Packet Data (CDPD) connection, digital satellite data connection or the like.

Many other devices or subsystems (not shown) may be connected in a similar manner (e.g., document scanners, digital cameras and so on). Conversely, all of the devices shown in FIG. 31 need not be present to practice the present systems and methods. The devices and subsystems can be interconnected in different ways from that shown in FIG. 31. The operation of a computer system such as that shown in FIG. 31 is readily known in the art and is not discussed in detail in this application. Code to implement the present disclosure can be stored in computer-readable medium such as one or more of system memory 717, fixed disk 744, optical disk 742, or floppy disk 738. The operating system provided on computer system 710 may be MS-DOS®, MS-WINDOWS®, OS/2®, UNIX®, Linux®, or another known operating system.

Moreover, regarding the signals described herein, those skilled in the art will recognize that a signal can be directly transmitted from a first block to a second block, or a signal can be modified (e.g., amplified, attenuated, delayed, latched, buffered, inverted, filtered, or otherwise modified) between the blocks. Although the signals of the above described embodiment are characterized as transmitted from one block to the next, other embodiments of the present systems and methods may include modified signals in place of such directly transmitted signals as long as the informational and/or functional aspect of the signal is transmitted between blocks. To some extent, a signal input at a second block can be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

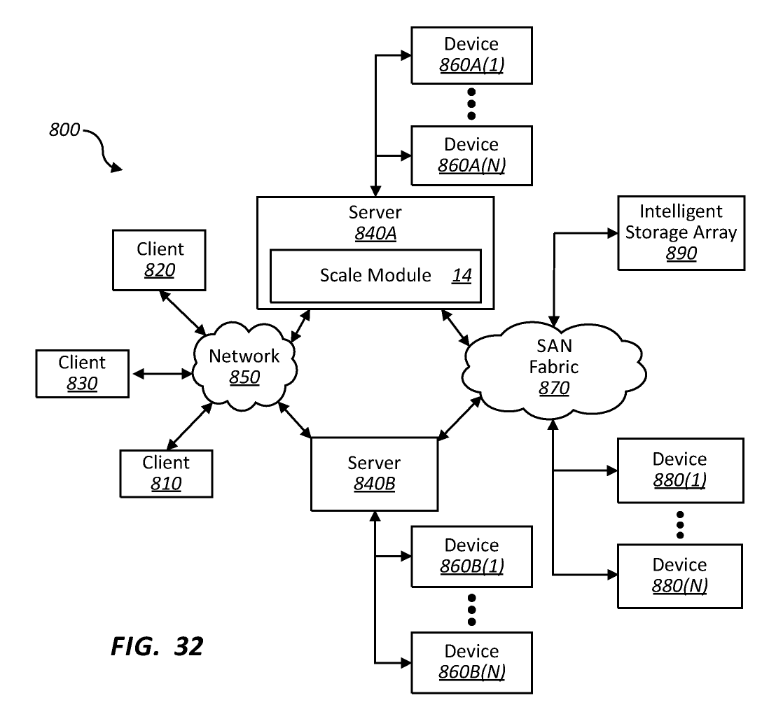

FIG. 32 is a block diagram depicting a network architecture 800 in which client systems 810, 820 and 830, as well as storage servers 840A and 840B (any of which can be implemented using computer system 810), are coupled to a network 850. In one embodiment, the labeling module 12 may be located within a storage server 840A, 840B to implement the present systems and methods. The storage server 840A is further depicted as having storage devices 860A(1)-(N) directly attached, and storage server 840B is depicted with storage devices 860B(1)-(N) directly attached. SAN fabric 870 supports access to storage devices 880(1)-(N) by storage servers 840A and 840B, and so by client systems 810, 820 and 830 via network 850. Intelligent storage array 890 is also shown as an example of a specific storage device accessible via SAN fabric 870.

With reference to computer system 710, modem 747, network interface 748 or some other method can be used to provide connectivity from each of client computer systems 810, 820, and 830 to network 850. Client systems 810, 820, and 830 are able to access information on storage server 840A or 840B using, for example, a web browser or other client software (not shown). Such a client allows client systems 810, 820, and 830 to access data hosted by storage server 840A or 840B or one of storage devices 860A(1)-(N), 860B(1)-(N), 880(1)-(N) or intelligent storage array 890. FIG. 32 depicts the use of a network such as the Internet for exchanging data, but the present systems and methods are not limited to the Internet or any particular network-based environment.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered exemplary in nature since many other architectures can be implemented to achieve the same functionality.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

Furthermore, while various embodiments have been described and/or illustrated herein in the context of fully functional computing systems, one or more of these exemplary embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the exemplary embodiments disclosed herein.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.”

What is claimed is:

1. A method of labeling medical ultrasound images, comprising:
selecting a type of medical ultrasound study;
selecting a probe orientation for the ultrasound study;
obtaining ultrasound images from a patient using a probe positioned at the selected probe orientation;
displaying the ultrasound images;
comparing the ultrasound images to a database of known ultrasound images within the selected study and probe orientation;
automatically labeling anatomical structures shown in the ultrasound images.

2. The method of claim 1, wherein labeling includes shading at least some of the anatomical structures.

3. The method of claim 1, wherein labeling includes outlining at least some of the anatomical structures.

4. The method of claim 1, wherein labeling includes adding at least one text label.

5. The method of claim 1, wherein the labeling appears and disappears as the anatomical structures move into and out of the ultrasound images.

6. The method of claim 1, wherein labeling includes displaying a list of labeling options and selecting at least one labeling option.

7. The method of claim 2, further comprising altering an opacity feature of the shading.

8. The method of claim 1, further comprising after selecting the type of study, displaying a menu of anticipated landmark anatomical structures to appear in the ultrasound images, and selecting which landmark structure to automatically label.

9. The method of claim 1, wherein after selecting the type of study, selecting from a plurality of ultrasonic views.

10. The method of claim 1, further comprising displaying a plurality of probe orientation diagrams and selecting among the displayed probe orientation diagrams.

11. The method of claim 1, wherein the ultrasound images are freeze frames of real-time ultrasound video.

12. A method of identifying and labeling medical ultrasound images, comprising:
providing an ultrasound probe, a display screen, a labeling module, and a database of ultrasound images;
collecting ultrasound images of anatomical structures from a patient with the ultrasound probe;
displaying the ultrasound images on the display screen;
referencing the database of ultrasound images to identify anatomical structures within the ultrasound images using the labeling module;
labeling the anatomical structures displayed on the display screen with the labeling module.

13. The method of claim 12, further comprising selecting a type of medical ultrasound study from a plurality of medical ultrasound studies displayed on the display screen.

14. The method of claim 13, further comprising selecting a probe orientation for the selected medical ultrasound study from a plurality of probe orientations displayed on the display screen.

15. The method of claim 12, wherein labeling includes shading at least some of the anatomical structures.

16. The method of claim 12, wherein labeling includes outlining at least some of the anatomical structures.

17. The method of claim 12, wherein labeling includes providing a text label for at least some of the anatomical structures.

18. The method of claim 12, further comprising automatically adding and removing the labeling as the anatomical structures move into and out of view on the display screen.

19. A method of labeling medical ultrasound images in real-time, comprising:
obtaining real-time ultrasound video of a patient;
taking ultrasound images from at least one frame of the ultrasound video;
displaying the ultrasound images;
comparing the ultrasound images to a database of known ultrasound images within the selected study and probe orientation;
automatically labeling anatomical structures shown in the ultrasound images.

20. The method of claim 19, wherein automatically labeling includes shading at least some of the anatomical structures.

21. The method of claim 19, wherein automatically labeling includes outlining at least some of the anatomical structures.

22. The method of claim 19, wherein automatically labeling includes adding at least one text label.

23. The method of claim 19, wherein the automatically labeling provides at least one label that appears and disappears as the anatomical structures move into and out view.

24. The method of claim 19, wherein automatically labeling includes displaying a list of labeling options and selecting at least one labeling option.

25. The method of claim 21, further comprising altering an opacity feature of the shading.

26. The method of claim 19, further comprising after selecting the type of study, displaying a menu of anticipated landmark anatomical structures to appear in the ultrasound images, and selecting which landmark structure to automatically label.

27. The method of claim 19, wherein after selecting the type of study, selecting from a plurality of ultrasonic views.

28. The method of claim 19, further comprising displaying a plurality of probe orientations and selecting among the displayed probe orientations.

29. The method of claim 19, further comprising selecting a type of medical ultrasound study.

30. The method of claim 19, wherein selecting a type of medical ultrasound study includes selecting a medical ultrasound study from a group consisting of:
Trauma exams;
Evaluation of an aorta for abdominal aortic aneurysms, dilation of aortic root, dissection, and acute occlusion;
Evaluation of shock states;
Evaluation of the hepatobiliary system for gallstones, gallbladder wall abnormalities, biliary inflammation and obstruction, pericholecystic fluid, common bile duct dilation;
Echocardiogram for pericardial effusion and tamponade, cardiac activity and contractility, heart chamber size, thrombus, detection of central venous volume status, and assessment of undifferentiated hypotension;
Evaluation of a thorax to identify pneumothorax, pleural fluid or hemothorax, pulmonary edema, pneumonia, inflammatory disorders, and trauma;
Identification of landmarks and vessels for central venous cannulation, peripheral vein cannulation, and arterial line cannulation;
Diagnosis of deep vein thrombosis;
Evaluation of deeper structures for localization of fluid collections or abscesses;
Diagnosis peri-tonsillar abscess;
Musculoskeletal and soft tissue exams (including evaluation of soft tissue infection for abscess, cellulitis, necrotizing fasciitis, identification of fractures, identification/removal of foreign bodies, evaluation of tears or injuries to muscles and tendons, identification of peripheral nerves for injuries and for anesthetic blocks, diagnosis of joint effusions or bursitis, evaluation of hip dysplasia, and identification of landmarks for lumbar puncture);
Abdominal ultrasound to identify peritoneal fluid, ascites or hemorrhage;
Evaluation of bowel obstruction, appendicitis, pyloric stenosis, and diverticulitis;
Evaluation of pregnancy for detection of intrauterine pregnancy, ectopic pregnancy, detection of fetal heart rate, dating of pregnancy, and detection of free fluid;
Evaluation of urinary tract for kidney or ureter stones, hydronephrosis, and bladder status;
Recognition of enlarged organs;
Pelvis ultrasound for ovarian torsion, cyst, mass, and uterine abnormalities;
Endoscopic ultrasound;
Scrotal ultrasound for evaluation of its contents and blood flow;
Evaluation of vasculature for dialysis catheters;
Evaluation of blood flow in neck vasculature;
Transcranial Doppler;
Evaluation of intracranial hemorrhage in newborns through the fontanel;
Identify esophageal versus tracheal intubations;
Ocular ultrasound to assess retinal detachments, hemorrhage, dislocations or ruptures, posterior chamber pathology, optic nerve sheath diameter, or other abnormalities;
Procedural guidance.

31. A system for labeling medical ultrasound images, comprising:
a processor;
an ultrasound probe;
a display screen configured to display ultrasound images collected by the ultrasound probe;
an ultrasound image database including a plurality of stored ultrasound images;
a labeling module configured to:
compare the ultrasound images displayed on the display screen to the stored ultrasound images;
automatically label the ultrasound images displayed on the display screen.

32. The system of claim 31, wherein the labeling module displays a list of medical ultrasound studies on the display screen for selection by a user.

33. The system of claim 31, wherein the labeling module displays a list of ultrasound probe orientations on the display screen for selection by a user.

34. The system of claim 33, wherein the list of ultrasound probe orientations includes diagrams for each ultrasound probe orientation.

35. The system of claim 31, wherein automatically labeling includes shading at least some of the anatomical structures.

36. The system of claim 31, wherein automatically labeling includes outlining at least some of the anatomical structures.

37. The system of claim 31, wherein automatically labeling includes providing a text label for at least some of the anatomical structures.

38. The system of claim 31, wherein the ultrasound images are still shots taken from a real-time ultrasound video.
